(19) United States
(12) Patent Application Publication
(10) Pub. No.: US 2014/0147336 A1
(43) Pub. Date: May 29, 2014

(54) IMPEDANCE BIOSENSOR FOR ELECTRICAL IMPEDANCE BIOLOGICAL SENSING AND MANUFACTURING METHOD THEREOF

(71) Applicant: NATIONAL CHI NAN UNIVERSITY, Puli (TW)

(72) Inventors: Tak-Sing CHING, Taichung City (TW); Tai-Ping SUN, Jhongli City (TW); Tzong-Ru CHOU, Puli (TW)

(73) Assignee: NATIONAL CHI NAN UNIVERSITY, Puli (TW)

(21) Appl. No.: 13/837,451

(22) Filed: Mar. 15, 2013

(30) Foreign Application Priority Data
Nov. 23, 2012 (TW) 101143940

(51) Int. Cl. G01N 33/543; H05K 3/00

(52) U.S. Cl. CPC G01N 33/54373; H05K 3/0011
USPC 422/69; 29/846

(57) ABSTRACT

An impedance biosensor for sensing concentration of a target analyte in a solution includes an insulator substrate, electrically coupled conductive trace units on the substrate, biological sensing films, and an insulator cover. Each trace unit has a first trace and a second trace, each having a sensing end portion and a connecting end portion. The biological sensing films are disposed on the sensing end portions, and have a capture layer for capturing the target analyte. The insulator cover covers the trace units and is formed with window openings that expose the sensing end portions and that cooperate with the insulator substrate to define a space for receiving the solution therein.

### CROSS-REFERENCE TO RELATED APPLICATION

This application claims priority to Taiwanese Application No. 101143940, filed on Nov. 23, 2012.

### BACKGROUND OF THE INVENTION

1. Field of the Invention

The invention relates to a biosensor and a manufacturing method thereof, and more particularly to an impedance biosensor and a manufacturing method thereof.

2. Description of the Related Art

Non-invasive bio-detection methods provide instant and comfortable selections for examinees, and include the following methods.

One method is label signal transmission detection. A target analyte is labeled with a fluorescent substance. The labeled target analyte receives light with a specific wavelength for exciting fluorescence, and the fluorescence intensity is sensed for analysis of the content of the target analyte.

Another method is surface plasmon resonance. A substance which can capture the target analyte is formed on a surface of a nano-gold particle layer to form a capture layer. After the target analyte is captured by the capture layer, the capture layer with the target analyte receives light, and the refraction of the reflected light is analyzed to obtain information of the content of the target analyte.

The two methods described above have high detection sensitivity and high precision in concentration range, but result in a high cost with complicated process for forming the capture layer. In addition, they require expensive light detection apparatuses with a great volume, such that they can only be used in research centers or hospitals.

Furthermore, the aforesaid capture layer is conventionally formed using self-assemble monolayer (SAM) technique, which must be processed on a nano-gold surface, a nano-silver surface, or a glass surface. Basically, it requires more than two chemical reaction procedures to coat the biomolecules on the substrate. The process spends a lot of time and money, and is difficult for quality control, resulting in difficulty of mass production.

Electrochemical signal transmission detection has been developed to coat a capture layer that captures the target analyte on an electrode surface for measuring variation of electrical signals (e.g., current or electrical impedance) through the capture layer with the target analyte to obtain information of the content of the target analyte. In addition, electrical signal detecting method provides possibility of miniaturization and portability of the biosensor. The blood glucose sensor is one of the commonly used products implementing this technique.

However, most researches of promoting sensitivity of the electrochemical signal transmission detection focus on links between the electrodes and the capture layer, structure of the capture layer, material of the capture layer, etc. As mentioned in “Anti-Prostate Specific Antigen (Anti-PSA) Modified Interdigitated Microelectrode-Based Impedimetric Biosensor for PSA Detection”, by Sunsil et al. in Biosensor Journal, 1(2012), pp. 1-7, an Anti-PSA is linked to gold electrodes in a single-electrode manner using covalent bonds for measuring the electrical impedance variation to obtain information of the content of the target analyte (Anti-PSA).

Based on the development of the aforesaid biosensors and consideration of miniaturization and portability, the applicants provide an impedance biosensor that is suitable for mass production with a low cost and a relatively simple manufacturing process, and that has good sensitivity.

### SUMMARY OF THE INVENTION

An object of the present invention is to provide an impedance biosensor that is small and easy to use, and that may provide instant detection and good sensitivity.

According to one aspect of the present invention, an impedance biosensor is adapted for sensing concentration of a target analyte in a solution, and comprises:

an insulator substrate having a surface;

a plurality of conductive trace units formed on the surface of the insulator substrate, each of the conductive trace units including a first trace and a second trace, each of the first and second traces having a sensing end portion and a connecting end portion, the conductive trace units being coupled electrically to each other;

a plurality of biological sensing films each disposed on a surface of a respective one of the sensing end portions of the first and second traces of the conductive trace units, and having a capture layer for capturing the target analyte; and

an insulator cover disposed on the insulator substrate to cover the conductive trace units and formed with a plurality of window openings, each of the window openings exposing the sensing end portions of the first and second traces of a respective one of the conductive trace units and cooperating with the substrate to define a space for receiving the solution therein.

Another object of the present invention is to provide a method for manufacturing an impedance biosensor with a relatively simple process and at a low cost.

According to another aspect of the present invention, a method for manufacturing an impedance biosensor comprises:

a) forming a plurality of conductive trace units on a surface of an insulator substrate, each of the conductive trace units including a first trace and a second trace, each of the first and second traces having a sensing end portion and a connecting end portion;

b) disposing an insulator cover on the insulator substrate to cover the conductive trace units, the insulator cover being formed with a plurality of window openings, each of the window openings exposing the sensing end portions of the first and second traces of a respective one of the conductive trace units, and cooperating with the substrate to define a space for receiving a solution that contains a target analyte;

c) forming a plurality of biological sensing films, each disposed on a surface of a respective one of the sensing end portions, each of the biological sensing films having a capture layer for capturing the target analyte; and

d) coupling electrically the conductive trace units to each other.

### BRIEF DESCRIPTION OF THE DRAWINGS

Other features and advantages of the present invention will become apparent in the following detailed description of the preferred embodiment with reference to the accompanying drawings, of which:

FIG. 1 is a schematic perspective view of a preferred embodiment of an impedance biosensor according to the present invention;

FIG. 2 is a flow chart illustrating a method for manufacturing the preferred embodiment;

FIG. 3 is an exploded view of the preferred embodiment illustrating a manufacturing process of the preferred embodiment;

FIG. 4 is a fragmentary schematic view showing a structure of the preferred embodiment;

FIG. 5 is a schematic diagram illustrating connection between the preferred embodiment and an impedance analyzer;

FIG. 6 is a plot showing impedance data measured using the preferred embodiment;

FIG. 7 is an enlarged view of a portion in FIG. 6;

FIG. 8 is a plot illustrating a relationship between impedance variation and target analyte concentration;

FIG. 9 is a schematic diagram showing single connection between the impedance biosensor and the impedance analyzer;

FIG. 10 is a plot showing impedance data measured using the preferred embodiment in a single connection manner; and

FIG. 11 is a histogram showing difference of SNR between the single connection manner and a preferred connection manner using the preferred embodiment.

### DETAILED DESCRIPTION OF THE PREFERRED EMBODIMENT

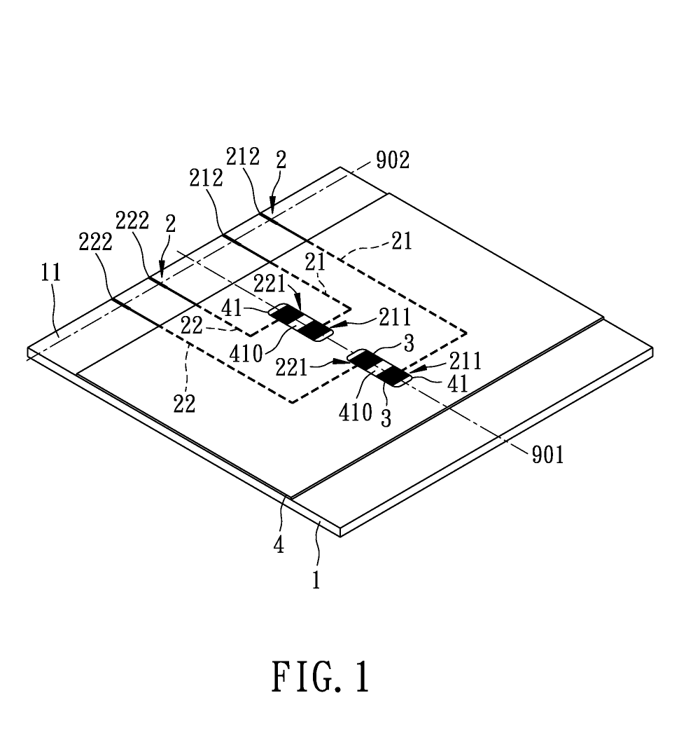

Referring to FIG. 1, the preferred embodiment of the impedance biosensor according to this invention is shown to include an insulator substrate 1, a plurality of conductive trace units 2, a plurality of biological sensing films 3 and an insulator cover 4, and is adapted for sensing a target analyte concentration in a solution. In this embodiment, the target analyte is an antigen.

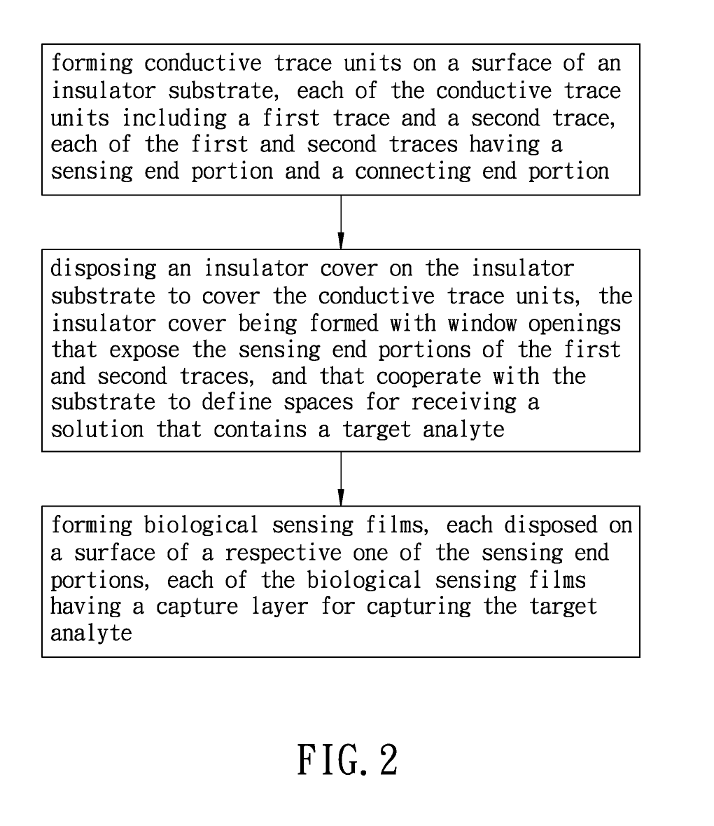

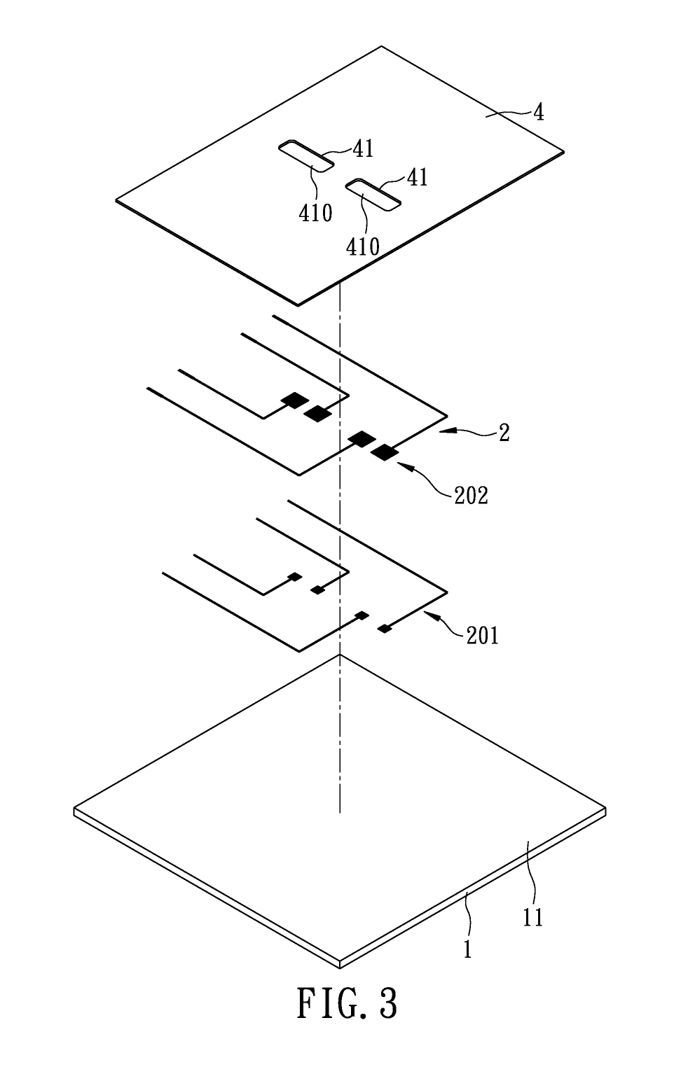

Further referring to FIGS. 2 and 3, the insulator substrate 1 is usually made of a polymer plastic material, such as polyethylene terephthalate (PET), and has a surface 11. The conductive trace units 2 are formed on the surface 11 of the insulator substrate 1 using screen printing. Each of the conductive trace units 2 includes a first trace 21 and a second trace 22. Each of the first traces 21 has a sensing end portion 211 and a connecting end portion 212. Each of the second traces 22 has a sensing end portion 221 and a connecting end portion 222.

In this embodiment, the impedance biosensor includes two conductive trace units 2. In detail, a silver paste layer 201 of the first and second traces 21, 22 is formed on the surface 11 of the insulator substrate 1, dried in a cool place, and baked at 70° C. for at least 30 minutes in an oven. Then, a carbon paste layer 202 of the first and second traces 21, 22 is formed on the silver paste layer 201, dried in the cool place, and baked at 25° C. for at least 15 minutes in the oven.

In the preferred embodiment, the sensing end portions 211, 221 of the first and second traces 21, 22 of the conductive trace units 2 are disposed along an imaginary line that extends in a first direction 901. The sensing end portions 211, 221 are spaced apart from each other in the first direction 901, and are distal from the connecting end portions 212, 222 of the first and second traces 21, 22 of the conductive trace units 2. The connecting end portions 212, 222 of the first and second traces 21, 22 of the conductive trace units 2 are arranged along a second direction 902 that is substantially perpendicular to the first direction 901. The first and second traces 21, 22 of the conductive trace units 2 are respectively disposed at two sides of the imaginary line in the first direction 901.

It should be noted that, when using the impedance biosensor of this invention, the conductive trace units 2 may be coupled electrically to each other with a jumper, direct connection by screen printing, etc. In this embodiment, the connecting end portions 222 of the second traces 22 of the two conductive trace units 2 are interconnected using a jumper to connect the conductive trace units 2 electrically in series. Through the series connection, signal strength may be promoted, resulting in higher signal-to-noise ratio (SNR).

After forming the conductive trace units 2, the insulator cover 4 is covered on the insulator substrate 1 to protect the conductive trace units 2. The insulator cover 4 is formed with a plurality of window openings 41. Each of the window openings 41 exposes the sensing end portions 211, 221 of the first and second traces 21, 22 of a respective one of the conductive trace units 2, and cooperates with the substrate 1 to define a space 410 for receiving the solution therein.

It should be noted that, in the preferred embodiment, the insulator cover 4 also exposes the connecting end portions 212, 222 of the first and second traces 21, for facilitating connection with an impedance analyzer.

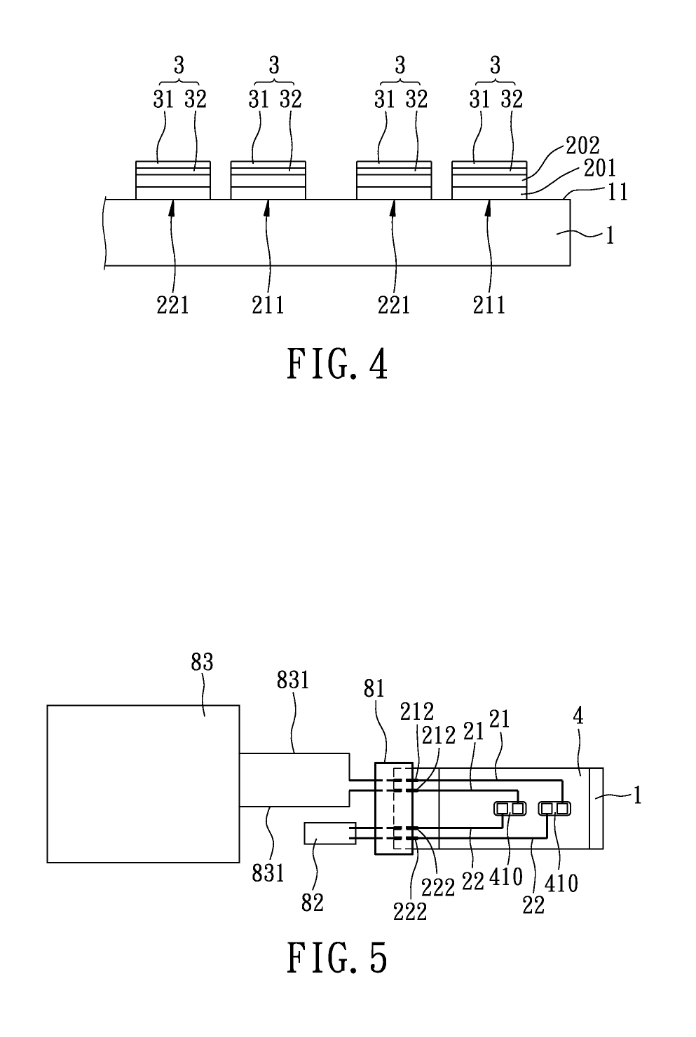

Further referring to FIG. 4, finally, for each of the sensing end portions 211, 221, a biological sensing film 3 is formed on a surface thereof. Each of the biological sensing films 3 has a capture layer 31 for capturing the target analyte. In this embodiment, the capture layer 31 includes an antibody that has immune reaction with the target analyte (antigen), resulting in impedance variation during electrical conduction.

In detail, a cross-linking agent is first filled in the spaces 410 through the window openings 41. In this embodiment, the cross-linking agent is a protein cross-linking agent. Then, an antibody solution is introduced into the spaces 410 for reacting with the cross-linking agent to form a cross-linking agent layer 32 of each of the biological sensing films 3 on the sensing end portions 211, 221, and to form the capture layer 31 of each of the biological sensing films 3 linked to the cross-linking agent layer 32. In further detail, the antibody included in the capture layer 31 of the preferred embodiment is a prostate specific antigen (PSA) antibody, and the protein cross-linking agent is a glutaraldehyde solution. The glutaraldehyde solution of 2.5% concentration is first filled in the spaces 410 with a same volume, and then the PSA antibody and bovine serum albumin-phosphate buffered solution (BSA-PBS) are filled for triggering the reaction. Then, the preferred embodiment is placed in a sealed space under 4° C. for one day to obtain the biological sensing films 3 that are linked to the surfaces of the sensing end portions 211, 221.

Therefore, instead of the SAM technique (as described in the prior art) that requires complicated chemical reaction procedures, the preferred embodiment uses simple protein cross-linking to link the capture layer 31 to the sensing end portions 211, 221 of the first and second traces 21, 22 through the cross-linking layer 32. Although the biological sensing film 3 in this invention itself may have fewer antibody and lower sensitivity, sensitivity of the impedance biosensor may be promoted through electrical connections between the conductive trace units 2, so as to result in a low cost, small volume, and good sensitivity of the impedance biosensor of this invention.

In this embodiment, the target analyte is PSA (available from Gwent Group of Companies, code: C2030519P4), the PSA antibody (available from GeneTex, catalog number: GTX28681) is a material of the capture layer 31, and impedance variation is measured using a precision impedance analyzer (available from Wayne Kerr Electronics, model: 6420). The following paragraphs describe the advantages of the impedance biosensor of this invention in application.

Referring to FIG. 5, the preferred embodiment of the impedance biosensor of this invention is installed on a reading connector platform 81 which has four pins respectively coupled to the connecting end portions 212, 222 of the first and second traces 21, 22. A jumper 82 is used to electrically interconnect the connecting end portions 222 of the second traces 22, so that the conductive trace units 2 are coupled in series. Two impedance analyzer clamps 831 of the impedance analyzer 83 are coupled to the connecting end portions 212 of the first traces 21, respectively.

First, a blank test was performed as follows. 10 μL of PBS was filled in both of the spaces 410. After 60 seconds, the impedance analyzer was activated to proceed with impedance measurement for 66 seconds using a MeterLinker program with the following parameter settings:

| | Frequency | Sam- | | Working | Primary | Secondary |
|---|---|---|---|---|---|---|
| Type | Range | pling | Unit | Voltage | Result | Result |
| Fre- | 20 Hz~10 | 100 | Hertz | 100 mV | Imped- | Phase |
| quency | MHz | points | (Hz) | | ance (Z) | Angle (θ) |

After obtaining basic impedance data (Zpbs) from the blank test, PBS used in the blank test was replaced by 10 μL of PSA solution at predetermined concentrations. A period of 3 minutes was allotted to allow the PSA to react with the capture layers 31 for linking with the biological sensing films 3. Then, the PSA was removed, and PBS was again filled in the spaces 410. After 60 seconds for stabilization, the impedance analyzer was activated to proceed with impedance measurement for 66 seconds to obtain antigen impedance data (Zpsa) for analysis of sensitivity and precision. The predetermined concentrations of the PSA solution were 6.25 ng/ml, 12.5 ng/ml, 50 ng/ml, and 200 ng/ml, respectively.

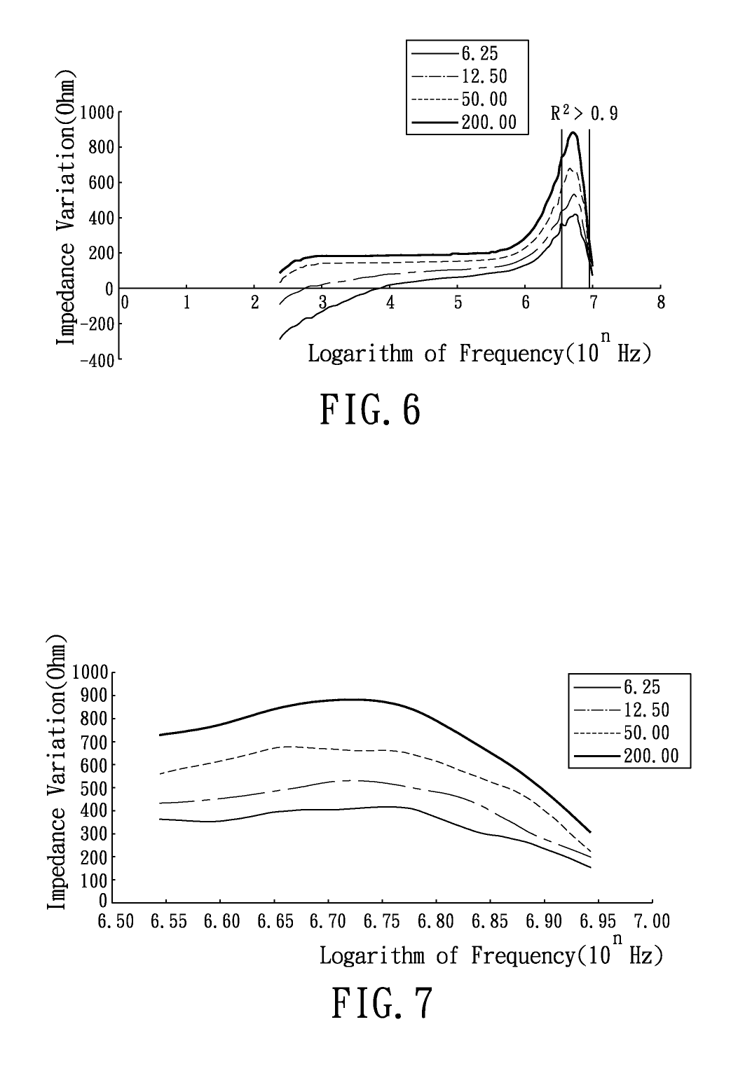

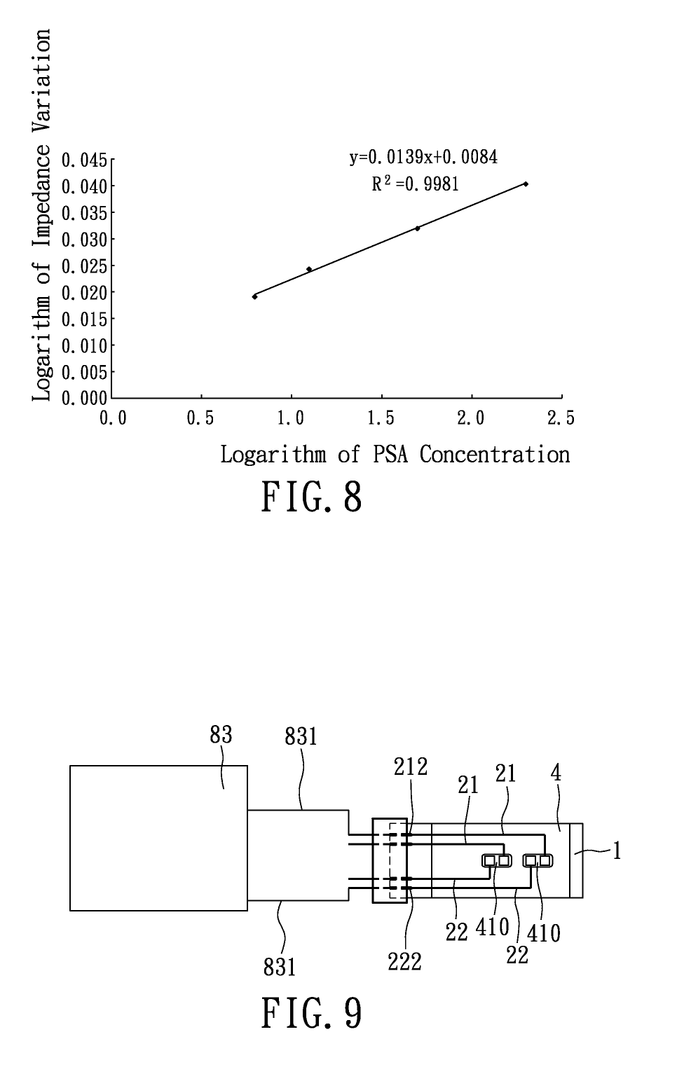

Referring to FIGS. 6 and 7, the frequency-impedance variation (difference between Zpsa and Zpbs) plot illustrates that the impedance variation varies between different analyte concentrations. Apparent differences between the different concentrations can be found in a high-frequency band between 4.55 MHz and 5.92 MHz. Through further analysis, it is found that, in this frequency band, there is a linear relationship between the impedance variation and the logarithm of the analyte concentration, and the coefficients of determination R² are greater than 0.9 within this frequency band. Further referring to FIG. 8, in this preferred embodiment, the optimal linear relationship appears at 4.55 MHz (R²=0.9981). In other words, the analyte concentration may be obtained by calculation using a pre-established linear equation with the measured impedance variation. The difference between the present invention and the conventional method resides in that the analysis frequency of this inventions falls within a high-frequency band, while the conventional biosensor that employs the SAM technique uses a low-frequency band.

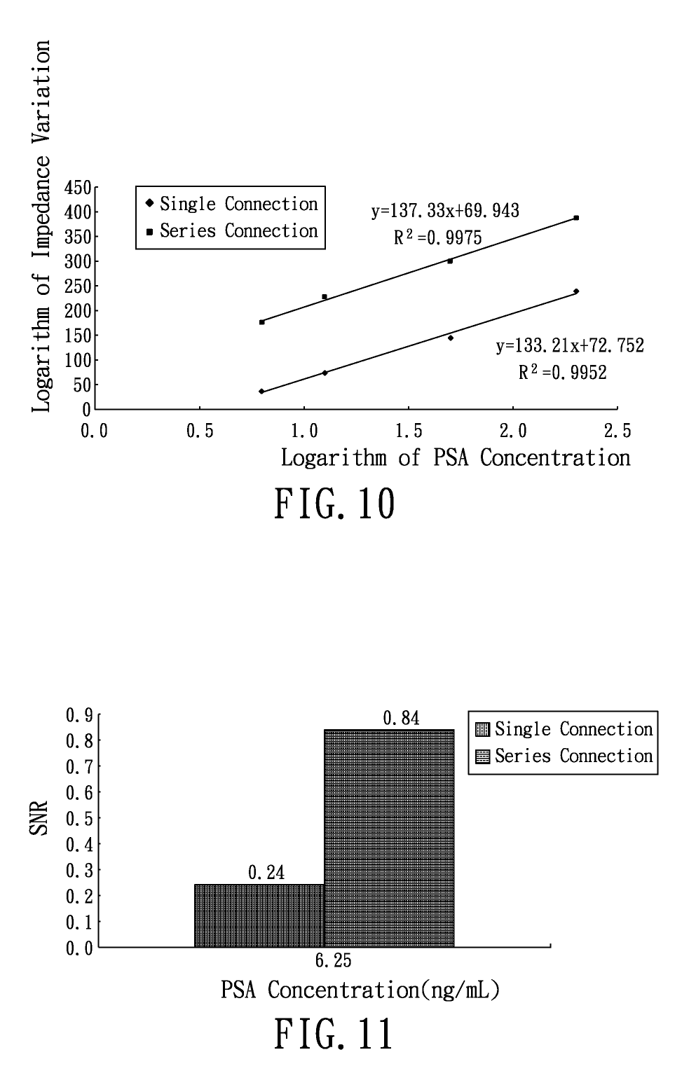

Referring to FIG. 9, for comparison, only one space 410 and one conductive trace unit 2 were used to proceed in a single connection manner. As shown in FIG. 10, the impedance measurement was performed at 1.59 MHz, and the electrical connection of this invention (series connection) results in better linearity and greater slope than those obtained in the single connection manner. The series connection of the present invention thus has better precision and sensitivity compared to the impedance biosensor using single connection.

Referring to FIG. 11, a SNR difference between the single connection and the series connection using the preferred embodiment is shown, in which the impedance was measured at 1.59 MHz and the analyte concentration was 6.25 ng/ml. The SNR is obtained using the equation of:

$$S N R = \frac{Signal ( Zpsa - Zpbs )}{Noise ( Zpbs )}$$

The SNR of the series connection is 0.84, while the SNR of the single connection is 0.24, which means that the present invention greatly promotes strength of the impedance signal. That is, sensitivity of the impedance biosensor is promoted.

To sum up, the present invention may be produced using screen printing, which is suitable for mass production, and the simple protein cross-linking reaction to obtain the impedance biosensor that is small and that may provide instant detection at a low cost. The impedance variation can be measured to calculate the target analyte concentration in a short time, so as to save time and cost required for inspection, and users may use the present invention at home for self-inspection and long term tracking.

While the present invention has been described in connection with what is considered the most practical and preferred embodiment, it is understood that this invention is not limited to the disclosed embodiment but is intended to cover various arrangements included within the spirit and scope of the broadest interpretation so as to encompass all such modifications and equivalent arrangements.

What is claimed is:

1. An impedance biosensor for sensing concentration of a target analyte in a solution, said impedance biosensor comprising:
an insulator substrate having a surface;
a plurality of conductive trace units formed on said surface of said insulator substrate, each of said conductive trace units including a first trace and a second trace, each of said first and second traces having a sensing end portion and a connecting end portion, said conductive trace units being coupled electrically to each other;
a plurality of biological sensing films each disposed on a surface of a respective one of said sensing end portions of said first and second traces of said conductive trace units, and having a capture layer for capturing the target analyte; and
an insulator cover disposed on said insulator substrate to cover said conductive trace units and formed with a plurality of window openings, each of said window openings exposing said sensing end portions of said first and second traces of a respective one of said conductive trace units and cooperating with said substrate to define a space for receiving the solution therein.

2. The impedance biosensor as claimed in claim 1, wherein said sensing end portions of said first and second traces of said conductive trace units are disposed along an imaginary line that extends in a first direction, are spaced apart from each other in the first direction, and are distal from said connecting end portions of said first and second traces of said conductive trace units.

3. The impedance biosensor as claimed in claim 2, wherein said connecting end portions of said first traces of said conductive trace units are arranged along a second direction that is substantially perpendicular to the first direction, and are spaced apart from each other in the second direction.

4. The impedance biosensor as claimed in claim 3, wherein said first traces of said conductive trace units are disposed at one side of the imaginary line, and said second traces of said conductive trace units are disposed at an opposite side of the imaginary line.

5. The impedance biosensor as claimed in claim 1, wherein said connecting end portions of said conductive trace units are interconnected to connect said conductive trace units electrically in series.

6. The impedance biosensor as claimed in claim 1, wherein the target analyte is an antigen, and the capture layer includes an antibody corresponding to the antigen.

7. The impedance biosensor as claimed in claim 1, wherein each of said first and second traces has a silver paste layer disposed on said surface of said insulator substrate, and a carbon paste layer covering said silver paste layer thereof.

8. The impedance biosensor as claimed in claim 1, wherein each of said biological sensing films further has a cross-linking agent layer to link said capture layer thereof to the corresponding one of said sensing end portions.

9. A method for manufacturing an impedance biosensor, comprising:
a) forming a plurality of conductive trace units on a surface of an insulator substrate, each of the conductive trace units including a first trace and a second trace, each of the first and second traces having a sensing end portion and a connecting end portion;
b) disposing an insulator cover on the insulator substrate to cover the conductive trace units, the insulator cover being formed with a plurality of window openings, each of the window openings exposing the sensing end portions of the first and second traces of a respective one of the conductive trace units, and cooperating with the substrate to define a space for receiving a solution that contains a target analyte;
c) forming a plurality of biological sensing films, each disposed on a surface of a respective one of the sensing end portions, each of the biological sensing films having a capture layer for capturing the target analyte; and
d) coupling electrically the conductive trace units to each other.

10. The method as claimed in claim 9, wherein, in step a), the conductive trace units are forming using screen printing.

11. The method as claimed in claim 9, wherein step a) includes:
forming a silver paste layer of the first and second traces on the surface of the insulator substrate, the sensing end portions of the first and second traces of the conductive trace units being disposed along an imaginary line that extends in a first direction, being spaced apart from each other in the first direction, and being distal from the connecting end portions of the first and second traces of the conductive trace units; and
forming a carbon paste layer of the first and second traces on the silver paste layer.

12. The method as claimed in claim 11, wherein the connecting end portions of the first traces of the conductive trace units are arranged along a second direction that is substantially perpendicular to the first direction, and are spaced from each other in the second direction.

13. The method as claimed in claim 12, wherein the first traces of the conductive trace units are disposed at one side of the imaginary line, and the second traces of the conductive trace units are disposed at an opposite side of the imaginary line.

14. The method as claimed in claim 9, wherein, in step d), the connecting end portions of the conductive trace units are interconnected to connect the conductive trace units electrically in series.

15. The method as claimed in claim 9, wherein the target analyte is an antigen, and the capture layer includes an antibody corresponding to the antigen.

16. The method as claimed in claim 15, wherein step c) includes:
filling the spaces with a cross-linking agent; and
introducing an antibody solution into the spaces for reacting with the cross-linking agent to form a cross-linking agent layer of each of the biological sensing films on the sensing end portions and to form the capture layer of each of the biological sensing films linked to the cross-linking agent layer.

17. The method as claimed in claim 16, wherein the cross-linking agent is a protein cross-linking agent.
